(19) United States
(12) Patent Application Publication
(10) Pub. No.: US 2016/0318829 A1
(43) Pub. Date: Nov. 3, 2016

(54) PREPARATION OF BUTADIENE BY OXIDATIVE DEHYDROGENATION OF N-BUTENE AFTER PRECEDING ISOMERIZATION

(71) Applicants: Felix GAERTNER, Haltern am See (DE); Guido STOCHNIOL, Haltern am See (DE); Joerg SCHALLENBERG, Dorsten (DE); Horst-Werner ZANTHOFF, Muelheim a.d. Ruhr (DE); Oliver Markus BUSCH, Recklinghausen (DE); Stephan PEITZ, Oer-Erkenschwick (DE); Frank GEILEN, Haltern am See (DE); Arne REINSDORF, Neubrandenburg (DE); Natalya PRODAN, Ratingen (DE)

(72) Inventors: Felix GAERTNER, Haltern am See (DE); Guido STOCHNIOL, Haltern am See (DE); Joerg SCHALLENBERG, Dorsten (DE); Horst-Werner ZANTHOFF, Muelheim a.d. Ruhr (DE); Oliver Markus BUSCH, Recklinghausen (DE); Stephan PEITZ, Oer-Erkenschwick (DE); Frank GEILEN, Haltern am See (DE); Arne REINSDORF, Neubrandenburg (DE); Natalya PRODAN, Ratingen (DE)

(73) Assignee: Evonik Degussa GmbH, Essen (DE)

(21) Appl. No.: 15/106,084

(22) PCT Filed: Dec. 4, 2014
(86) PCT No.: PCT/EP2014/076569

§ 371 Date: Jun. 17, 2016

(30) Foreign Application Priority Data
Dec. 18, 2013 (DE) 10 2013 226 370.8

(51) Int. Cl. C07C 5/48; B01J 38/12; B01J 23/887; C07C 5/25; B01J 23/02

(52) U.S. Cl. CPC C07C 5/48; C07C 5/2512; B01J 23/02; B01J 23/8876; B01J 38/12; C07C 2521/04; C07C 2523/02; C07C 2523/887; B01J 2523/00

(57) ABSTRACT

The invention relates to a process for preparing 1,3-butadiene by heterogeneously catalysed oxidative dehydrogenation of n-butene, in which a butene mixture comprising at least 2-butene is provided. The problem that it addresses is that of specifying a process for economically viable preparation of 1,3-butadiene on the industrial scale, which is provided with a butene mixture as raw material, wherein the 1-butene content is comparatively low compared to the 2-butene content thereof, and in which the ratio of 1-butene to 2-butene is subject to variation. This problem is solved by a two-stage process in which, in a first stage, the butene mixture provided is subjected to a heterogeneously catalysed isomerization to obtain an at least partly isomerized butene mixture, and in which the at least partly isomerized butene mixture obtained in the first stage is then subjected, in a second stage, to oxidative dehydrogenation. The two-stage process leads to higher butadiene yields compared to the one-stage process.

The invention relates to a process for preparing 1,3-butadiene by heterogeneously catalysed oxidative dehydrogenation of n-butene, in which a butene mixture comprising at least 2-butene is provided.

1,3-Butadiene (CAS no. 106-99-0) is an important commodity chemical in the chemical industry. It is the starting component in important polymers having various possible uses, including the sector of the automotive industry.

As well as 1,3-butadiene, 1,2-butadiene also exists, but the latter is of little interest because of its low industrial significance. Where reference is made here to “butadiene” or “BD” for short, what is meant is always 1,3-butadiene.

A general introduction into the chemical and physical properties of butadiene and preparation thereof can be found in:
Grub, J. and Löser, E. 2011. Butadiene. Ullmann's Encyclopedia of Industrial Chemistry.

At present, butadiene is usually obtained industrially by extractive separation from C₄ streams. C₄ streams are mixtures of different hydrocarbons having four carbon atoms, which are obtained in mineral oil crackers as coproducts in ethylene and propylene production.

In the future, global demand for butadiene will rise in the face of increasing scarcity of butadiene-containing C₄ streams. The reason is an altered raw materials situation and restructuring of refinery processes.

An alternative method for controlled and coproduct-free production of butadiene is the oxidative dehydrogenation (ODH) of n-butene.

The butenes are the four isomeric substances 1-butene, cis-2-butene, trans-2-butene and isobutene. 1-Butene and the two 2-butenes belong to the group of the linear butenes, while isobutene is a branched olefin. The linear C₄ olefins 1-butene, cis-2-butene and trans-2-butene are also referred to as “n-butene”.

An overview of the chemical and physical properties of the butenes and of the industrial workup and utilization thereof is given by:
Obenaus, F., Droste, W. and Neumeister, J. 2011. Butenes. Ullmann's Encyclopedia of Industrial Chemistry.

Just like butadiene, butenes are obtained in the cracking of mineral oil fractions in a steamcracker or in a fluid catalytic cracker (FCC). However, the butenes are not obtained in pure form but as what is called a “C₄ cut”. This is a mixture of hydrocarbons having four carbon atoms that has a different composition depending on its origin, and which comprises not only C₄ olefins but also saturated C₄ hydrocarbons (alkanes). In addition, traces of hydrocarbons having more or fewer than four carbon atoms (for example, but not exclusively, propane and/or pentenes) and other organic or inorganic accompanying substances may be present. Alternative sources of butenes are, for example, chemical processes such as the dehydrogenation of butane, ethylene dimerization, metathesis, methanol-to-olefin methodology, Fischer-Tropsch, and the fermentative or pyrolytic conversion of renewable raw materials.

Since butadiene-containing C₄ streams are becoming increasingly scarce, research at present is increasingly concentrating on the production of butadiene by the route of oxidative dehydrogenation from butenes.

Jung et al. Catal. Surv. Asia 2009, 13, 78-93 describe a multitude of mixed transition metal oxides, especially ferrites or bismuth molybdates, which are suitable as heterogeneous catalysts for ODH.

US2012130137A1 also describes a bismuth molybdate over which butene-containing streams can be oxidatively dehydrogenated with an oxygenous gas to give butadiene.

In order to optimally utilize available raw material sources, there have been descriptions of processes in which the oxidative dehydrogenation of butene to butadiene is used together with other reactions in a multistage process concept.

For example, WO2006075025 or WO2004007408A1 describes a process which couples an autothermally catalysed, nonoxidative dehydrogenation of butane to butene with an oxidative dehydrogenation of the butenes obtained to give butadiene. This opens up a direct route for the preparation of butadiene from butane, which is little utilized industrially in chemical conversions except for the preparation of maleic anhydride. The disadvantage of this process is large recycling streams as a result of recycling butane, which increase the apparatus and operating costs.

By means of the process described in US20110040134, it is also possible to use isobutene-containing streams for the oxidative dehydrogenation of butene to butadiene. This is enabled by a skeletal isomerization of isobutene to 2-butene that precedes the oxidative dehydrogenation. The disadvantage of this process is that it is based on isobutene, a raw material that can be used in other ways with a greater addition of value. The preparation of butadiene from isobutene is therefore uneconomic.

The butene isomers 1-butene and 2-butene can be converted over different catalysts at different rates to give butadiene (WO2009119975). By layering a double fixed catalyst bed, the overall yield can be improved significantly compared to a comparative experiment with only one catalyst type. In the examples mentioned, ferrite and mixed bismuth/molybdenum oxide catalysts are used. However, different optimal operating conditions of the catalysts lead to different industrial lifetimes of individual catalysts, which entails comparatively frequent interruption of operation for exchange of the individual catalysts.

U.S. Pat. No. 3,479,415 describes a process in which 2-butene-containing streams are converted via an isomerization and subsequent separation step to 1-butene. The distillatively enriched 1-butene is subsequently converted in an oxidative dehydrogenation stage to butadiene. A disadvantage is the additional energy-intensive separation step for preparation of enriched 1-butene. Moreover, 1-butene is a raw material having a comparable addition of value potential to 1,3-butadiene, and so the processing of 1-butene to butadiene makes barely any sense in economic terms.

Of greater economic interest is the preparation of butadiene from n-butene mixtures which, as well as 1-butene, also contain a high proportion of 2-butene.

A process for direct utilization of such streams in butadiene preparation is described in EP2256101A2. The oxidative dehydrogenation of the n-butene present in the input stream is effected in a double fixed bed comprising two different catalyst systems. The first catalyst is a bismuth molybdate over which the 1-butene present in the butene mixture is converted to butadiene. The conversion of the 2-butene is catalysed using a zinc-ferrite system. It is an undisputed advantage of this process that it allows the direct utilization of input mixtures which comprise not only 1-butene but also 2-butene. It is a disadvantage of this process that the two 2-butenes are less reactive compared to 1-butene, and therefore the residence time of the n-butenes in the double fixed bed is unnecessarily long: here, the slower reaction determines the process duration. The higher the proportion of 2-butene compared to 1-butene, the greater the adverse effect. Therefore, the process is tied to a restricted 1-butene to 2-butene ratio, in order to achieve sufficiently high n-butene conversions. If variable raw material sources afford butene mixtures having a variable ratio of 1-butene to 2-butene, losses in the butadiene yield have to be accepted in this process.

In the light of this prior art, the problem addressed by the invention is that of specifying a process for economically viable preparation of 1,3-butadiene on the industrial scale, which is provided with a butene mixture as raw material wherein the 1-butene content is comparatively low compared to the 2-butene content thereof, in which the ratio of 1-butene to 2-butene is subject to variation and in which the absolute contents of 1-butene and 2-butene also change over time. In short, butadiene is to be prepared with a high yield from a difficult raw material.

This problem is solved by a two-stage process in which, in a first stage, the butene mixture provided is subjected to a heterogeneously catalysed isomerization to obtain an at least partly isomerized butene mixture, and in which the at least partly isomerized butene mixture obtained in the first stage is then subjected, in a second stage, to oxidative dehydrogenation.

The invention therefore provides a process for preparing 1,3-butadiene by heterogeneously catalysed oxidative dehydrogenation of n-butene, in which a butene mixture comprising at least 2-butene is provided, in which the butene mixture provided is subjected to a heterogeneously catalysed isomerization to obtain an at least partly isomerized butene mixture, and in which the at least partly isomerized butene mixture is then subjected to oxidative dehydrogenation.

First of all, one basic idea of the present invention is to improve the overall process of butadiene preparation by replacing the 2-butenes, which are of comparatively low reactivity, with much more reactive 1-butene. This is done by first converting 2-butene present in the starting mixture by way of a double bond isomerization to 1-butene, and by supplying the butene mixture which is now enriched in terms of its 1-butene content to the oxidative dehydrogenation. This dispenses with an additional costly separation process for enrichment of 1-butene between the two steps.

The enrichment of 1-butene caused by the isomerization in the butene mixture which is to be fed into the ODH leads to a better space-time yield, since 1-butene is more reactive than 2-butene.

The isomerization, especially in the case of utilization of feeds having varying n-butene composition, results in an advantage since it balances out the varying ratio of 1-butene to 2-butene. For this reason, the two-stage process proposed here displays its advantages over a one-stage process specifically when a “difficult” butene mixture is being provided, wherein the 1-butene and 2-butene content is unfavourable and also varies.

According to the invention, the mixture need not be isomerized fully, i.e. need not be isomerized as far as the thermodynamic equilibrium of 1-butene and 2-butene. It may also be sufficient to shift the isomer distribution in the direction of the equilibrium, without reaching equilibrium. For this reason, the isomerization, according to the invention, should be conducted at least partially, but not necessarily fully as far as equilibrium.

According to whether the thermodynamic equilibrium in the butene mixture provided has been shifted in the direction of 1-butene or in the direction of 2-butene, there is a conversion in the course of the isomerization of 1-butene to 2-butene or of 2-butene in the direction of 1-butene.

One embodiment of the invention accordingly envisages that the isomerization is effected in such a way that 2-butene present in the butene mixture provided is isomerized to 1-butene, such that the 1-butene content in the at least partly isomerized butene mixture has increased compared to the butene mixture provided.

In contrast, in a second embodiment of the invention, the isomerization is effected in such a way that 1-butene present in the butene mixture provided is isomerized to 2-butene, such that the 1-butene content in the at least partly isomerized butene mixture has decreased compared to the butene mixture provided.

The at least partly isomerized butene mixture obtained from the isomerization is preferably transferred directly, i.e. without further purification, into the ODH. Before the at least partly isomerized butene mixture is subjected to the oxidative dehydrogenation, therefore, no components are separated out of the mixture. This saves energy.

The higher butadiene yield is also achieved through the choice of a catalyst optimized for the particular reaction stage. Accordingly, an isomerization catalyst is provided for the first reaction stage (isomerization), while the oxidative dehydrogenation (second stage) is effected in the presence of a specific dehydrogenation catalyst. The optimization of the respective catalysts will generally result in the use of non-identical catalysts for isomerization and for oxidative dehydrogenation. Accordingly, a preferred development of the invention envisages that the isomerization catalyst and the dehydrogenation catalyst are not identical.

Useful isomerization catalysts are in principle all catalysts which catalyse the double bond isomerization of 2-butene to 1-butene. In general, these are mixed oxide compositions comprising aluminium oxide, silicon oxide, and mixtures and mixed compounds thereof, zeolites and modified zeolites, aluminas, hydrotalcites, borosilicates, alkali metal oxides or alkaline earth metal oxides, and mixtures and mixed compounds of the components mentioned. The catalytically active materials mentioned may additionally be modified by oxides of the elements Mg, Ca, Sr, Na, Li, K, Ba, La, Zr, Sc, and oxides of the manganese group, iron group and cobalt group. The metal oxide content, based on the overall catalyst, is 0.1% to 40% by weight, preferably 0.5% to 25% by weight.

Suitable isomerization catalysts are disclosed, inter alia, in DE3319171, DE3319099, U.S. Pat. No. 4,289,919, U.S. Pat. No. 3,479,415, EP234498, EP129899, U.S. Pat. No. 3,475,511, U.S. Pat. No. 4,749,819, U.S. Pat. No. 4,992,613, U.S. Pat. No. 4,499,326, U.S. Pat. No. 4,217,244, WO03076371 and WO02096843.

In a particularly preferred form, the isomerization catalyst comprises at least two different components, the two components having been mixed with one another or the first component having been applied to the second component. In the latter case, the catalyst is frequently of the type known as a supported catalyst, in which the first component constitutes the essentially catalytically active substance, while the second component functions as support material. However, some catalysis experts express the view that the support of a conventional supported catalyst is likewise catalytically active. For this reason, reference is made in this context, without any consideration of any catalytic activity, to a first component and a second component.

Very particularly suitable isomerization catalysts have been found to be two-component systems comprising an alkaline earth metal oxide on an acidic aluminium oxide support or a mixture of Al₂O₃ and SiO₂. The alkaline earth metal oxide content, based on the overall catalyst, is 0.5% to 30% by weight, preferably 0.5% to 20% by weight. Alkaline earth metal oxides used may be magnesium oxide and/or calcium oxide and/or strontium oxide and/or barium oxide.

The second component (i.e. “support”) used is aluminium oxide or silicon dioxide or a mixture of aluminium oxide and silicon dioxide or an aluminosilicate.

A catalyst which is based on MgO and aluminosilicate and is suitable for isomerization is described in EP1894621B1.

A system of even better suitability as isomerization catalyst is that known from EP0718036A1, in which strontium oxide as first component has been applied to aluminium oxide as second component. The strontium content here is between 0.5% and 20% by weight, based on the total catalyst weight. Alternatively, it is possible to use a heterogeneous catalyst in which magnesium oxide as first component has been mixed with an aluminosilicate as second component. Catalysts of this kind are disclosed in EP1894621A1.

Catalysts used for the oxidative dehydrogenation may in principle be all the catalysts suitable for the oxidative dehydrogenation of n-butene to butadiene. Two catalyst classes in particular are useful for this purpose, namely mixed metal oxides from the group of the (modified) bismuth molybdates, and also mixed metal oxides from the group of the (modified) ferrites.

Very particular preference is given to using catalysts from the group of the bismuth molybdates, since these convert 1-butene to butadiene more quickly than 2-butene in the oxidative dehydrogenation. In this way, the effect caused by an isomerization of 2-butene to 1-butene conducted beforehand is manifested to a particular degree.

Bismuth molybdates are understood to mean catalysts of formula (I)

(Moₐ Bi_{b} Fe_{c} (Co+Ni)_{d} Dₑ E_{f} F_{g} Gₕ Hᵢ) Oₓ (I)

in which
D: at least one of the elements from W, P,
E: at least one of the elements from Li, K, Na, Rb, Cs, Mg, Ca, Ba, Sr,
F: at least one of the elements from Cr, Ce, Mn, V,
G: at least one of the elements from Nb, Se, Te, Sm, Gd, La, Y, Pd, Pt, Ru, Ag, Au,
H: at least one of the elements from Si, Al, Ti, Zr
   and
the coefficients a to i represent rational numbers selected from the following ranges, including the specified limits:
a=10 to 12
b=0 to 5
c=0.5 to 5
d=2 to 15
e=0 to 5
f=0.001 to 2
g=0 to 5
h=0 to 1.5
i=0 to 800
   and
x is a number which is determined by the valency and frequency of the elements other than oxygen.

Catalysts of this kind are obtained, for example, by the preparation steps of co-precipitation, spray drying and calcination. The powder obtained in this way can be subjected to a shaping operation, for example by tableting, extrusion or coating of a support. Catalysts of this kind are described in U.S. Pat. No. 8,003,840, U.S. Pat. No. 8,008,227, US2011034326 and in U.S. Pat. No. 7,579,501.

By-products which may be formed during the isomerization include traces of coke deposits, isobutene, isobutane and butadiene. According to the process conditions of the isomerization, it is also possible for traces of saturated and unsaturated C₁ to C₃ products to arise, and also higher-boiling saturated and unsaturated compounds, especially C₈ compounds, and also coke and coke-like compounds. The deposition of coke on the isomerization catalyst causes the continuous deactivation thereof. However, the activity of the isomerization catalyst can be very substantially re-established by regeneration, for example by burning off the deposits with oxygenous gas.

The dehydrogenation catalyst can be deactivated in a similar manner. Regeneration of the dehydrogenation catalysts is possible by oxidation with an oxygenous gas. The oxygenous gas may be air, technical grade oxygen, pure oxygen or oxygen-enriched air. However, dehydrogenation catalysts are deactivated much more slowly than isomerization catalysts. Isomerization catalysts, in contrast, have to be reactivated quite frequently.

In order to avoid interruptions to operation caused by the regeneration of the catalysts, there are different conceivable process designs which simultaneously enable the performance of the particular intended reaction and the regeneration of the catalyst. Specifically, the isomerization can be effected continuously in an isomerization arrangement of the following specification:
a) the isomerization arrangement comprises a reaction zone and a regeneration zone;
b) the isomerization is effected within the reaction zone of the isomerization arrangement in the presence of isomerization catalyst disposed in the reaction zone of the isomerization arrangement;
c) there is simultaneous regeneration of isomerization catalyst disposed in the regeneration zone of the isomerization arrangement, especially by burning off deposits on the isomerization catalyst with an oxygenous gas;
d) there is continuous exchange of isomerization catalyst between the reaction zone and the regeneration zone of the isomerization arrangement.

In this design, the reactivation of the catalyst is effected with spatial separation from the reaction zone. This has the advantage that the installation space for the regeneration zone can be reduced, since the regeneration is effected much more rapidly than the deactivation. The disadvantage of this design is that it requires a continuous exchange of the catalyst between the regeneration zone and the reaction zone, which has to be accomplished by a suitable conveying means. This increases the susceptibility of the plant to faults.

If the installation space for the plant does not constitute the limiting factor, it is possible to resort to the following design, which is reliable in terms of operation, for regeneration of the isomerization catalysts without interrupting operation:
a) the isomerization arrangement comprises two universal zones, each of which is utilizable either as reaction zone or as regeneration zone;
b) one of the two universal zones is utilized as reaction zone for isomerization, while the other universal zone is being utilized as regeneration zone for regeneration of the isomerization catalyst;
c) the isomerization is effected within the universal zone utilized as reaction zone in the presence of isomerization catalyst disposed in the reaction zone;
d) there is simultaneous regeneration of isomerization catalyst disposed in the universal zone utilized as regeneration zone, especially by burning off deposits on the isomerization catalyst with an oxygenous gas.

In this design, two universal zones are accordingly used, in each of which both the isomerization and the regeneration of the isomerization catalyst can be conducted. Both universal zones are charged with isomerization catalyst which remains in the respective zones. In this way, it is possible, without interrupting operation, to regenerate the catalyst in one universal zone, while isomerization is conducted in the other universal zone.

In the simplest case, the respective functions of the universal zones are switched cyclically. The disadvantage of cyclical switching is that the regeneration zone is inactive after conclusion of the regeneration, until the deactivation of the catalyst in the reaction zone requires a switch of function. The reason for this is that the regeneration proceeds more quickly than the deactivation, and that the universal zones each require the full reactor volume. In this way, costly reactor installation space is regularly inactive.

In order to avoid this, an isomerization arrangement with two universal zones can be operated as follows:

Until a particular level of deactivation of the isomerization catalyst is attained, the two universal zones are utilized in parallel as reaction zones. Then one of the two universal zones is utilized as regeneration zone, while the other universal zone is still being utilized as a reaction zone. When the catalyst has been fully reactivated again in the regeneration zone, the other universal zone is put into regenerative operation. Then the two zones are utilized as reaction zones again. In this design, the regeneration is of course commenced at a lower level of deactivation than in the cyclical switching design. The advantage of this process is the cost-saving continuous utilization of the entire design space of the two universal zones and of the entire mass of catalyst.

Technical configurations for performance of a continuous process procedure with continuous regeneration of the catalysts used will be explained in detail later on.

Depending on the quality of the butene mixture provided, it is even possible to entirely dispense with a complex regeneration design. As soon as the isomerization catalyst has been deactivated, the entire isomerization arrangement is simply bypassed, such that the butene mixture provided is passed into the ODH without prior isomerization. While the process is effectively operated in one stage, the regeneration is effected in the sole universal zone of the isomerization arrangement. Because of the isomerization that does not occur during the regeneration, losses in the butadiene yield have to be accepted. Advantageously, the regeneration is conducted in periods in which the butene mixture, which varies in terms of its composition, has a 1-butene/2-butene ratio favourable for the ODH.

Incidentally, the dehydrogenation catalyst can be reactivated in the same way as outlined above for the isomerization catalyst. However, this will not be necessary, since dehydrogenation catalysts are deactivated much more slowly than isomerization catalysts. For this reason, the plant, after deactivation of the dehydrogenation catalyst, is simply shut down and the dehydrogenation catalyst is regenerated in situ or exchanged.

The butadiene to be produced is in a product mixture which results from the oxidative dehydrogenation. The product mixture comprises, as well as the butadiene target product, unconverted constituents of the butene mixture and unwanted by-products of the oxidative dehydrogenation. More particularly, the product mixture comprises, according to the reaction conditions and composition of the butene mixture provided, butane, nitrogen, residues of oxygen, carbon monoxide, carbon dioxide, water (steam) and unconverted butene. In addition, the product mixture may contain traces of saturated and unsaturated hydrocarbons, aldehydes and acids. In order to separate the desired butadiene from these unwanted accompanying components, the product mixture is subjected to a butadiene removal, in the course of which 1,3-butadiene is separated from the other constituents of the product mixture.

For this purpose, the product mixture is preferably first cooled and quenched with water in a quench column. With the aqueous phase thus obtained, water-soluble acids and aldehydes, and also high boilers, are removed. The product mixture thus prepurified, after a possible compression, passes into an absorption/desorption step or into a membrane process for removal of the hydrocarbons having four carbon atoms present therein. The butadiene can be obtained from this desorbed C₄ hydrocarbon stream, for example, by extractive distillation.

The butadiene removal is not restricted to the process variant described here. Alternative isolation methods are described in the article in Ullmann cited at the outset.

A preferred development of the invention then envisages recycling of a portion of the product mixture and mixing with the butene mixture provided and/or at least partly isomerized butene mixture. In this way, materials of value that are thus far unconverted can be subjected again to the isomerization and/or the ODH. What is recycled is a quantitative portion of the product mixture obtained from the ODH and/or a physical portion of the product mixture, for instance a residue from the butadiene removal depleted of butadiene.

Preferably, a C₄ hydrocarbon stream obtained from the butadiene removal is recycled prior to the isomerization and/or the oxidative dehydrogenation, in order to convert the butene unconverted in the first pass to butadiene.

The reaction conditions for isomerization and/or the oxidative dehydrogenation preferably have the following values:
temperature: 250° C. to 500° C., especially 300° C. to 420° C.
pressure: 0.08 to 1.1 MPa, especially 0.1 to 0.8 MPa
weight hourly space velocity (g(butenes)/g(active catalyst composition)/h): 0.1 h⁻¹ to 5.0 h⁻¹, especially 0.15 h⁻¹ to 3.0 h⁻¹

In this context, the temperature means the temperature which is established in the reactor apparatus. The actual reaction temperature may differ therefrom. However, the reaction temperature, i.e. the temperature measured at the catalyst, will likewise be within the ranges specified.

More preferably, the two reactions take place at similar temperatures and pressures, because it is thus possible to dispense with energy-intensive intermediate compression or decompression, or heating and cooling, of the at least partly isomerized butene mixture. Energy-intensive purification between the two stages is likewise not required. Especially the use of a strontium-containing catalyst based on aluminium oxide for isomerization, and of a bismuth molybdate-containing catalyst as a dehydrogenation catalyst, allows the energy-saving performance of the two reaction steps under similar operating conditions.

The oxidative dehydrogenation is preferably performed in the presence of an inert gas such as nitrogen and/or steam. A preferred embodiment of the invention envisages metered addition of steam and also of the oxygen required for the oxidative dehydrogenation after the isomerization, and accordingly feeding thereof into the stream downstream of the isomerization. In this way, the stream through the isomerization becomes smaller, which lowers the apparatus costs associated with the reactor volume.

The proportion of steam in the mixture supplied to the dehydrogenation is preferably 1 to 30 molar equivalents based on the sum total of 1-butene and 2-butene, preferably 1 to 10 molar equivalents based on the sum total of 1-butene and 2-butene. The oxygen content in the mixture supplied to the dehydrogenation is preferably 0.5 to 3 molar equivalents based on the sum total of 1-butene and 2-butene, preferably 0.8 to 2 molar equivalents based on the sum total of 1-butene and 2-butene. The sum total of all the proportions of different substances in % by volume adds up to a total proportion of 100% by volume.

The process according to the invention is exceptionally suitable for processing of input mixtures containing a small proportion of 1-butene. It is possible to use any streams containing 2-butene as a utilizable substrate. The butene mixture is preferably provided in gaseous form.

Generally, suitable input mixtures are C₄ hydrocarbon streams of any kind, in which no hydrocarbons having more or fewer than four carbon atoms are present in proportions exceeding 10% by weight.

Preference is given to providing, as the input mixture, butene-containing streams in which the 1-butene concentration is below the thermodynamic equilibrium concentration of 1-butene at the temperature in the isomerization, based on n-butene. Preferably, the butene mixture provided has a butane content between 0% and 90% by weight, while the n-butene content is between 5% and 100% by weight. Particular preference is given to using streams in which the 2-butene concentration is between 5% and 100% by weight. As well as n-butene and butane, it is also possible for other alkanes and alkenes to be present in proportions of less than 5% by weight. This applies especially to isobutene, isobutane, propane, propene, neopentane, neopentene and butadiene. In addition, the butene mixture provided may also comprise other secondary components, for example oxygen-containing components such as steam, water, acids or aldehydes, and sulphur-containing components, for example hydrogen sulphide or other sulphides, nitrogen-containing components, for example nitriles or amines.

More preferably, the butene mixture provided has the following specification:
a) the proportion by weight of hydrocarbons having four carbon atoms, based on the overall butene mixture provided, is at least 90%;
b) the total proportion by weight of n-butane and isobutane, based on the overall butene mixture provided, is 0% to 90%;
c) the total proportion by weight of isobutene, 1-butene, cis-2-butene and trans-2-butene, based on the overall butene mixture provided, is 5% to 100%;
d) the total proportion by weight of cis-2-butene and trans-2-butene, based on the butene content of the butene mixture provided, is 5% to 100%.

The percentages outlined here, of course, always add up to 100%.

Preference is given to providing, as raw material, butene mixtures having a variable content of 1-butene and 2-butene over time. Butene mixtures of this kind are quite expensive because the utilizability thereof is problematic. Since the process according to the invention, even in the case of a variable 1-butene/2-butene ratio, achieves a high butadiene yield, the value that it adds to such raw material sources is particularly high. It is also possible to use mixtures in which not just the isomer ratio but also the absolute content of 1-butene and 2-butene varies.

Various sources are possible for the butene mixture provided. It is possible to utilize either C₄ streams from naphtha crackers or raffinates which are obtained during the utilization of such C₄ streams. More particularly, it is possible to use what is called “raffinate III” as input stream. Raffinate III in this context is understood to mean a C₄ hydrocarbon stream which originates from a naphtha cracker and from which the butadiene, isobutene and 1-butene have already been removed. Raffinate III contains almost exclusively 2-butene as olefinic product of value, which can be converted with the aid of the present process to higher-value 1,3-butadiene.

It is also possible to use, as input stream, butene mixtures which have been obtained by oxidative or nonoxidative dehydrogenation of butane mixtures. An example of a useful butane mixture is liquefied petroleum gas (LPG).

It is likewise possible to use, as input mixture, butene-containing streams which are prepared by fluid catalytic cracking (FCC) of mineral oil fractions. Streams of this kind are increasingly replacing the crack C4 that originates from naphtha crackers, but contain barely any 1,3-butadiene. The present process is consequently suitable for preparation of butadiene from FCC C4.

For the sake of completeness, it is pointed out that the butene mixtures used can also originate from C₂ dimerization reactions such as ethylene dimerization. It is also possible to use those butene-containing streams which are prepared by dehydration of 1-butanol or 2-butanol. Of course, the butene mixture provided may also be a mixture of the above-described C₄ sources. Finally, it is also possible to feed a recycled material from upstream process steps into the butene mixture provided, such as, more particularly, a portion of product mixture that has been freed of butadiene. It is also possible to recycle streams immediately beyond the isomerization. It is also possible to add the steam or oxygen required in the oxidative dehydrogenation directly to the butene mixture provided. Finally, it is also possible to utilize ethane crackers, which afford barely any butadiene, as a raw material source for provision of the butene mixture. Further sources for suitable input mixtures are, for example, chemical processes such as the dehydrogenation of butane, ethylene dimerization, metathesis, methanol-to-olefin methodology, Fischer-Tropsch, and the fermentative or pyrolytic conversion of renewable raw materials. It is also possible to use C₄ streams that originate from processes which are operated for enrichment and/or depletion of particular C₄ isomers. The enrichment or depletion can be effected by absorptive or adsorptive means, or by membrane separation. One example of an absorptive separation is butadiene extraction, the C₄-containing output from which is called “raffinate I”. A further absorptive process whose output can be used as input mixture is the BUTENEX process. An adsorptive process whose output can be utilized as input stream is the OLE-SIV process.

The present invention will now be illustrated in detail by working examples. The figures show, in schematic form:

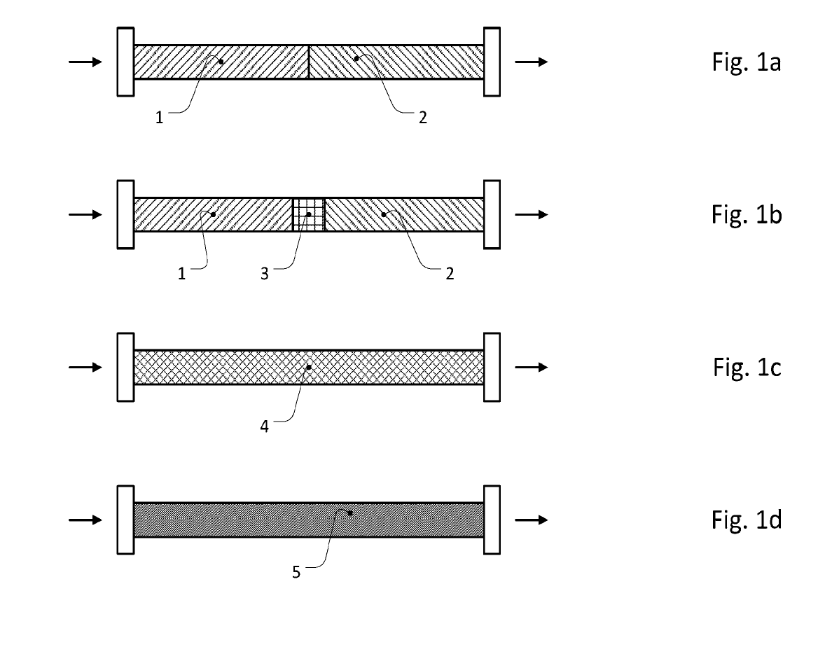

FIG. 1a: process in a double fixed bed;

FIG. 1b: process in a double fixed bed with an inert bed arranged in between;

FIG. 1c: process in a single fixed bed consisting of a physical mixture of two catalyst systems;

FIG. 1d: process in a single fixed bed consisting of a universal catalyst;

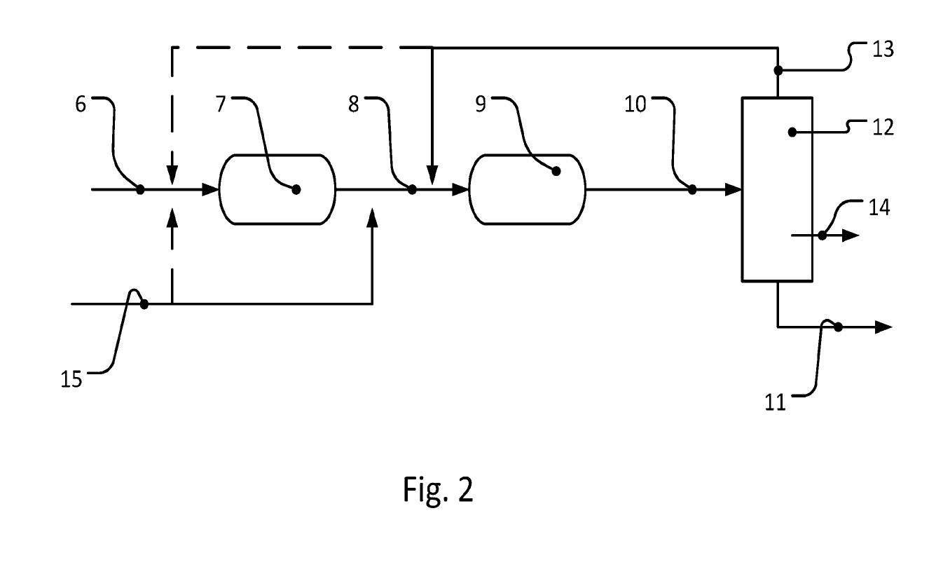

FIG. 2: simplified process flow diagram;

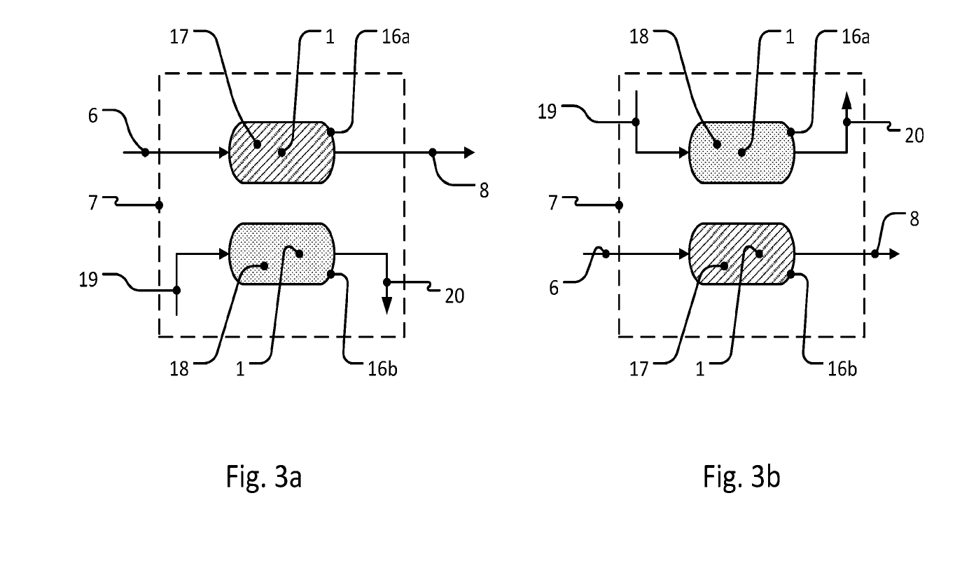

FIGS. 3a and b: operating states of an isomerization arrangement comprising two universal zones in cyclical operation;

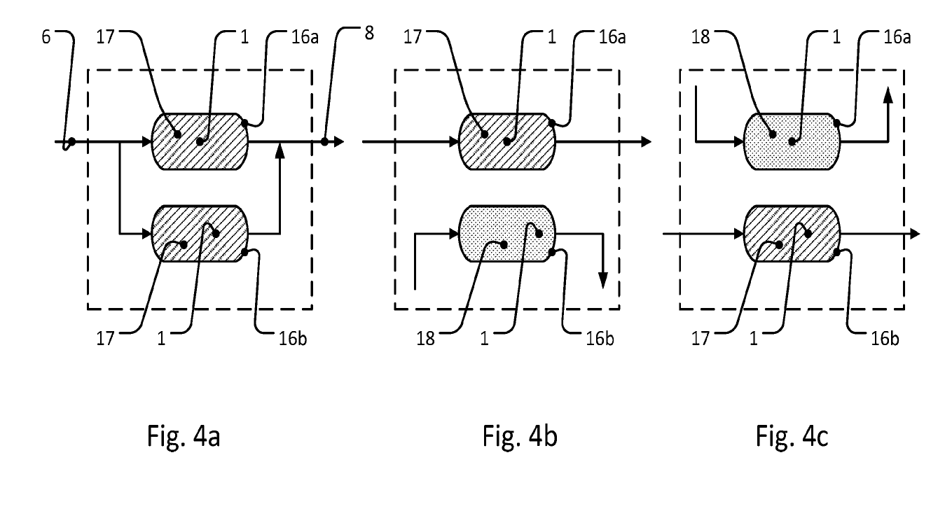

FIGS. 4a to c: operating states of an isomerization arrangement comprising two universal zones in parallel operation;

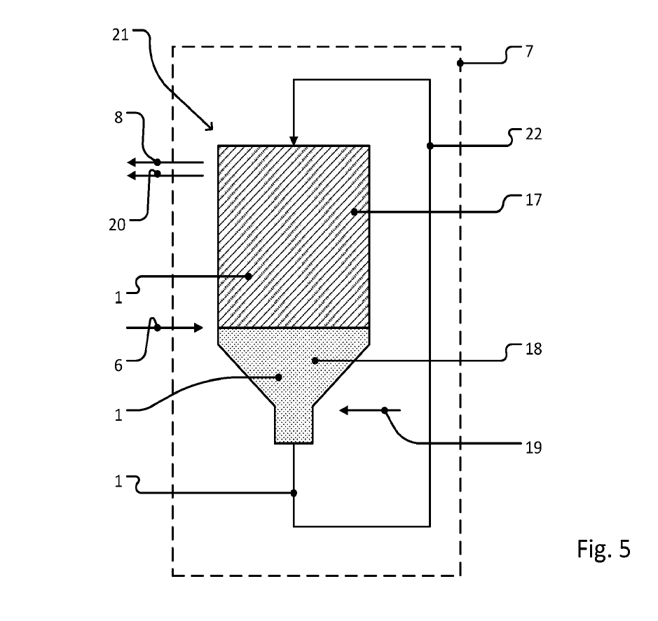

FIG. 5: isomerization arrangement in the form of a fluidized bed reactor;

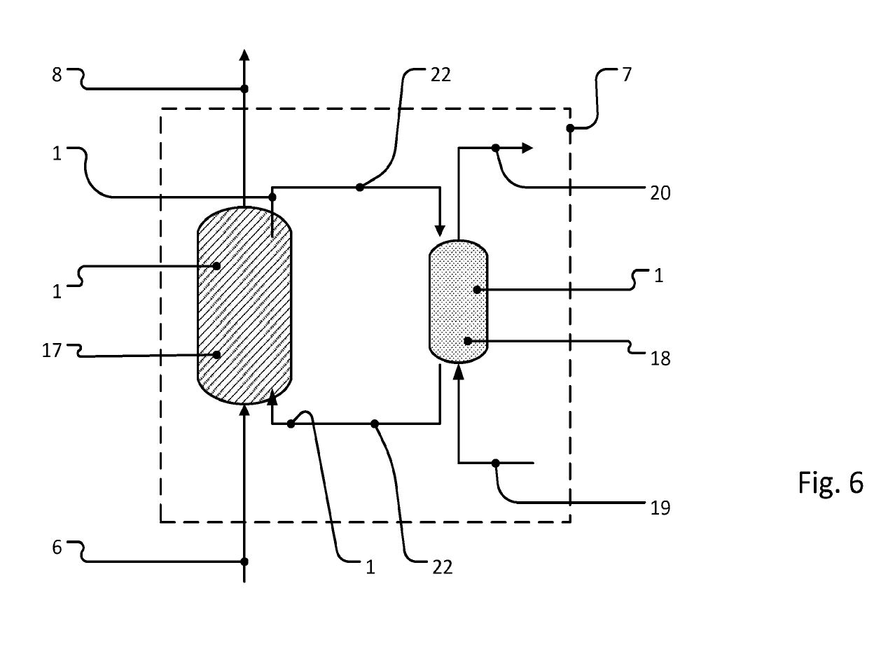

FIG. 6: isomerization arrangement comprising two fluidized bed reactors;

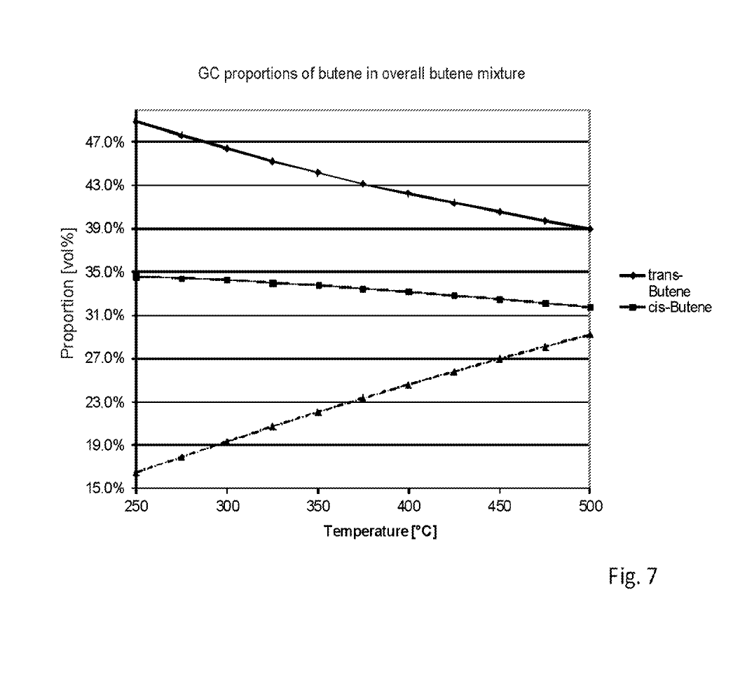

FIG. 7: representation of the thermodynamic equilibrium concentration of 1-butene in a mixture with 2-butenes as a function of temperature.

The process according to the invention comprises two essential steps, namely first the double bond isomerization of the 2-butene present in the butene mixture provided to 1-butene and, thereafter, the oxidative dehydrogenation of the butene mixture enriched in 1-butene in the first step to give butadiene. FIGS. 1a to 1d show different catalyst designs in schematic form.

In the variant shown in FIG. 1a, the process is conducted with two catalysts having different specialisms, namely with an isomerization catalyst 1 and a dehydrogenation catalyst 2. Both catalysts are heterogeneous fixed bed catalysts which together form a double fixed bed.

In order to prevent mixing of the two catalyst beds during operation, it is optionally possible to undertake a spatial separation of the two beds, for example by means of an inert bed 3 or a sieve tray (FIG. 1b).

In the embodiment shown in FIG. 1c, a single fixed bed consisting of a physical mixture 4 of isomerization catalyst and dehydrogenation catalyst is used. The ODH preferentially converts the 1-butene component and, as a result, removes it from the isomerization equilibrium, such that further 2-butene can permanently react to give 1-butene.

FIG. 1d shows a further single fixed bed which, however, does not consist of two catalysts but is formed by a universal catalyst 5 which both isomerizes and dehydrogenates. The advantage of this embodiment is that only one kind of catalyst bed has to be introduced into the reactor.

All the fixed beds shown in FIGS. 1a to 1d are arranged in a tubular reactor, and the material flows through them in these figures from left to right.

FIG. 2 shows the schematic setup of one possible embodiment of the plant for performance of the process, using a simplified process flow diagram.

First of all, a butene mixture 6 is provided and transferred into an isomerization arrangement 7 in which the butene mixture 6 provided is subjected to an isomerization. This at least partly isomerizes 2-butene present in the butene mixture 6 provided to 1-butene, in such a way that the 1-butene content in the isomerized butene mixture 8 drawn off from the isomerization arrangement 7 has been increased. In the simplest case, the isomerization is effected up to the thermodynamic equilibrium at the temperature that prevails in the isomerization arrangement, i.e. to completion. It may also be advantageous not to conduct the isomerization to completion, but to conduct it only partially. In that case, the isomer distribution is not yet completely at the thermodynamic equilibrium, but is more balanced than before the isomerization. If the isomer distribution of the butene mixture provided is biased in the direction of 1-butene, meaning that it contains too little 2-butene, the isomerization leads to an increase in the 2-butene content in the at least partly isomerized butene mixture 8.

The partly or fully isomerized butene mixture 8 is transferred into a dehydrogenation arrangement 9 in which the 1-butene and 2-butene present in the isomerized butene mixture 8 is oxidatively dehydrogenated. A product mixture 10 is drawn off from the dehydrogenation arrangement 9 and may comprise, as well as the desired butadiene, also unconverted reactants and further accompanying substances in the butene mixture 6 provided. In addition, the product mixture 10 may contain by-products formed in the isomerization 7 and the dehydrogenation 9.

In order to separate butadiene 11 from the product mixture 10, the product mixture 10 is transferred into a butadiene removal 12. Within the butadiene removal 12, the target butadiene product 11 is removed, so as to obtain a butadiene-depleted residue 13 of the product mixture 10. This residue 13 can be recycled into one of the preceding steps, for example by mixing with the at least partly isomerized butene mixture 8 and/or by mixing with the butene mixture 6 provided.

In order to avoid the enrichment of unwanted by-products such as, more particularly, high boilers in the process, it is possible for by-products to leave the process via a discharge stream 14 in the course of butadiene removal 12.

For the performance of the oxidative dehydrogenation 9, an oxygen stream 15 is required as further reactant, and is preferably added to the isomerized butene mixture 8. In the same way, steam can also be added to the isomerized butene mixture 8. Alternatively, the oxygen-containing stream 15 and steam can also be added to the butene mixture 6 provided. The oxygen can be fed in in the form of pure oxygen, as an air mixture or with oxygen-enriched air. It should be ensured here that explosive mixtures are not formed.

FIGS. 3a and 3b show, in schematic form, the design of an isomerization arrangement 7 provided with two universal zones 16a and 16b. The two universal zones 16a, 16b have been filled with isomerization catalyst 1. The two universal zones 16a, 16b are each utilizable either as a reaction zone 17 or as a regeneration zone 18. In the operating state shown in FIG. 3a, the first universal zone 16a is utilized as a reaction zone, in such a way that butene mixture 6 provided is subjected to an isomerization therein, such that an isomerized butene mixture 8 is drawn off from the reaction zone 17.

At the same time, a regeneration of the isomerization catalyst 1 present in the second universal zone 16b takes place therein. For this purpose, the isomerization catalyst 1 is contacted with an oxygenous gas 19, in order to burn off deposits such as, more particularly, coke from the isomerization catalyst 1. The offgases 20 formed are disposed of. The regeneration of the isomerization catalyst 1 conducted in the regeneration zone 18 proceeds more quickly than the deactivation of the isomerization catalyst present in the first universal zone 16a, which is utilized for isomerization as intended. For this reason, on conclusion of the regeneration, the stream with the oxygenous gas 19 is shut down, while the isomerization continues in the reaction zone 17. This operating state is not shown in the drawings.

As soon as the deactivation of the isomerization catalyst 1 present in the first universal zone 16 a has progressed, the operating state shown in FIG. 3b is established. For this purpose, the first universal zone 16a is utilized as regeneration zone 18, while the isomerization proceeds in the second universal zone 16b. Isomerization catalyst is not exchanged for this purpose between the two universal zones 16a and 16b. In practical operation, the switch between the two operating states 3a and 3b is effected according to fixed cycles, the duration of which is judged by experience.

The disadvantage of the cyclical mode of operation shown in FIGS. 3a and 3b is that the regeneration zone 18 is unutilized as soon as the regeneration has concluded, but the deactivation in the reaction zone 17 has not yet progressed to such an extent that regeneration would be required. A way in which the valuable reactor volume of an isomerization arrangement 7 having two universal zones 16a, 16b can be exploited better is shown in FIGS. 4a to 4c:

At first, the two universal zones 16a, 16b are operated in parallel as reaction zone 17 (FIG. 4a). As soon as the deactivation has progressed to such an extent that regeneration is worthwhile, just one universal zone 16b is switched to regenerative operation (FIG. 4b). The other universal zone 16 a continues to be operated as a reaction zone 17. Because the feed is now larger here, the deactivation of the isomerization catalyst present in the first universal zone 16 a now proceeds more quickly. However, the regeneration of the isomerization catalyst 1 present in the second universal zone 16b is also concluded rapidly, such that the freshly regenerated isomerization catalyst 1 in the second universal zone 16b can now be utilized for isomerization, while regeneration is then effected in the other universal zone 16a (FIG. 4c). On conclusion of this regeneration, both universal zones 16a, 16b are again operated in parallel as reaction zones 17 (FIG. 4a).

An alternative to the use of two universal zones is shown in FIG. 5. The isomerization arrangement 7 shown therein is executed industrially by a fluidized bed reactor 21. The fluidized bed reactor 21 is set up vertically and is divided into a reaction zone 17 and a regeneration zone 18. The regeneration zone 18 is arranged beneath the reaction zone 17. The fluidized bed reactor 21 is filled completely with isomerization catalyst 1 through both zones 17, 18.

The butene mixture 6 provided is blown in at the base of the reaction zone 17, ascends, is subjected to the isomerization, and leaves the top of the fluidized bed reactor as an isomerized butene mixture 8. Beneath the reaction zone 17 is the regeneration zone 18. At the base thereof, oxygenous gas 19 is blown in, ascends, and regenerates the isomerization catalyst 1 present in the regeneration zone 18. The offgas 20 formed as a result leaves the fluidized bed reactor together with the isomerized butene mixture 8.

At the base of the fluidized bed reactor 21, isomerization catalyst 1 is drawn off continuously in the freshly regenerated state and applied again at the top of the fluidized bed reactor 21 by means of a conveying device 22. The isomerization catalyst 1 then slides from the top downward through the reaction zone 17 and then through the regeneration zone 18. In this way, a continuous circulation of regeneration catalyst 1 in countercurrent to the butene mixture 6 provided or to the oxygenous gas 19 arises. Just like the volumes of the reaction zone 17 and of the regeneration zone 18, the circulation rate should be such that the residence times of the isomerization catalyst 1 in the respective zones 17, 18 correspond to the deactivation and regeneration periods thereof.

A further alternative to the continuous, parallel operation of regeneration and reaction is shown by the isomerization arrangement 7 shown in schematic form in FIG. 6. This comprises a reaction zone 17 and a regeneration zone 18 which are spatially separate from one another. The two zones 17 and 18 can be configured either as fluidized bed reactors or as moving bed reactors, and filled with isomerization catalyst 1. Possible fluidized bed reactors are of any kind known in industry, for example including bubble-forming fluidized beds, riser, downers, etc. It is also possible to use those fluidized beds in which spent catalyst is constantly replaced by fresh catalyst from outside. This is necessary in the case of particularly severe abrasion.

In the reaction zone, there is continuous isomerization of butene mixture 6 provided to at least partly isomerized butene mixture 8. The regeneration of the spent isomerization catalyst 1 is effected in the regeneration zone 18 by contacting of the deactivated isomerization catalyst 1 with oxygenous gas 19, which, after passing through the regeneration zone 18, is drawn off as offgas 20. If the regeneration zone 18 takes the form of a fluidized bed regenerator, the oxygenous gas 19 can be used as fluidization medium. Equally, the butene mixture 6 provided can be used as fluidization medium if the reaction zone 17 takes the form of a fluidized bed reactor. Continuous exchange of spent and freshly regenerated isomerization catalyst 1 between the two zones 17, 18 is effected by means of a constantly operated conveying device 22.

Catalyst stream and feed stream may flow in countercurrent or cocurrent in the two zones 17 and 18; in all the embodiments, the zones 17 and 18 can be operated at different temperatures.

Although different embodiments of an isomerization arrangement 7 have been elucidated in FIGS. 3, 4, 5 and 6, it should be made clear that a dehydrogenation arrangement can also be executed in the same way. However, the regeneration of the ODH catalyst is not absolutely necessary, since the dehydrogenation catalyst in reality has a lifetime of about three years and consequently need not be regenerated periodically. If regeneration is in fact necessary, there is a switch from a reaction mode to a regeneration mode at irregular intervals. The dehydrogenation arrangement consequently needs only a single universal zone.

In a particular embodiment of the invention, the butene mixture 6 provided has a 1-butene content below the thermodynamic equilibrium concentration of 1-butene which arises from the temperature that exists in the isomerization and/or in the oxidative dehydrogenation. The thermodynamic equilibrium concentration of 1-butene in a mixture of 1-butene with 2-butene can be seen in FIG. 7: within the particularly preferred temperature interval for isomerization and dehydrogenation between 300 and 420° C., the equilibrium concentration of 1-butene is between 21% by volume and 25.5% by volume. The proportion of 1-butene within the n-butene fraction in the butene mixture 6 provided is lower in the particularly preferred embodiment.

The processing of butene mixtures of constantly varying composition is particularly demanding. The variations are balanced out by the isomerization, such that the process according to the invention is outstandingly suitable for preparation of valuable butadiene from relatively low-value streams.

### EXAMPLES

Composition of the provided butene mixture used:
n-butane: 69.4% by vol.
cis-2-butene: 9.0% by vol.
trans-2-butene: 20.0% by vol.
1-butene: 1.6% by vol.

### Procedure for Isomerization/ODH Experiments (Examples 1a, 2a, 3a, 4a)

The experiments for two-stage isomerization/ODH were conducted in a laboratory apparatus which comprised two tubular reactors arranged in series. The first reactor (ISO zone) was charged with isomerization catalyst, and the second reactor (ODH zone) with mixed BiMo oxide catalyst. Between the two reaction zones, it was possible to add steam and air to the isomerized C₄ mixture leaving the isomerization zone.

The provided C₄ mixture introduced into the first reaction zone, without further dilution, was subjected to an isomerization of the 2-butene present in the C₄ mixture provided to 1-butene at a reactor temperature of 380° C.

The 1-butene concentration was determined during the examples by means of GC analysis downstream of the ISO zone. The isomerized C₄ mixture which leaves the ISO zone at 380° C. contained, during all the examples described here, 20.0% by volume±0.4% by volume of 1-butene (based on the n-butene mixture of trans-2-, cis-2- and 1-butene), which is well above the 1-butene concentration that the C4 mixture provided has (5.2% by volume of 1-butene, based on the n-butene mixture of trans-2-, cis-2- and 1-butene). Over a service life of 1100 h, there was no observation of any degree of deactivation of the isomerization catalyst that necessitated a regeneration.

The isomerized C4 mixture formed in the ISO zone was subsequently mixed with steam and air and then introduced into the second tubular reactor (ODH zone). The temperature of the second tubular reactor was varied in steps of 10° C. within the range of 360-390° C. The molar ratios of O₂ (from air)/n-butene/steam in the feed introduced into the ODH zone were 1/1/4. After departure from the ODH zone, the amount of butadiene formed in the product mixture was determined by means of GC analysis.

Summary of process parameters in ISO zone:
Temperature: 380° C.
Catalyst: 1-2 mm extrudates of 8% SrO on Al₂O₃, described in DE4445680
Weight hourly space velocity: 0.8 g_{n-butene}/g_{catalyst}/h

Feed: The pure C₄ mixture provided was isomerized

Summary of process parameters in ODH zone
Temperature: Individual experiments at 360-390 ° C.
Catalyst: Co_{5.1}Ni_{3.1}Fe_{1.78}Bi_{1.45}Mo₁₂ described in U.S. Pat. No. 8,008,227
Weight hourly space velocity: 0.8 g_{n-butene}/g_{catalyst}/h

Feed: steam and air were added to the isomerized C₄ mixture from the isomerization zone before it was introduced into the ODH zone. The molar ratios of O₂ (from air)/butene/steam in the feed introduced into the ODH zone were 1/1/4.

### Procedure for Comparative Experiment (Counter-Examples 1b, 2b, 3b, 4b): ODH Without Prior Isomerization

The comparative experiments were conducted in a similar test apparatus in which no ISO zone was present. The C₄ mixture provided was not subjected to any isomerization, and was mixed directly with steam and air and fed to the ODH zone. The molar ratios of O₂ (from air)/n-butene/steam in the feed introduced into the ODH zone were 1/1/4.

The yield of butadiene formed was determined by means of GC analysis in an analogous manner to the ISO/ODH examples. Apart from the absence of the ISO zone, all the other process parameters were identical to those in the ISO/ODH examples.

Summary of process parameters in ODH zone
Temperature: Individual experiments at 360-390° C.
Catalyst: Co_{5.1}Ni_{3.1}Fe_{1.78}Bi_{1.45}Mo₁₂ described in U.S. Pat. No. 8,008,227
Weight hourly space velocity: 0.8 g_{n-butene}/g_{catalyst}/h

Feed: the C4 mixture provided was mixed with steam and air and fed to the ODH zone.

The molar ratios of O₂ (from air)/n-butene/steam in the feed introduced into the ODH zone were 1/1/4.

### Overview of Results of the ISO/ODH Experiments and Counter-Example (Pure ODH)

| | | | ISO | ODH | n-Butene | Butadiene | Butadiene |
|---|---|---|---|---|---|---|---|
| | | | temperature | temperature | conversion | yield | selectivity |
| Example no. | ISO_ODH | ODH | [° C.] | [° C.] | [%] | [%] | [%] |
| 1a | x | | 380 | 380 | 93.2 | 83.0 | 89.0 |
| 1b (counter- | | x | | 380 | 94.8 | 77.1 | 81.3 |
| example) | | | | | | | |
| 2a | x | | 380 | 370 | 92.7 | 84.0 | 90.6 |
| 2b (counter- | | x | | 370 | 93.1 | 78.4 | 84.3 |
| example) | | | | | | | |
| 3a | x | | 380 | 360 | 91.8 | 83.7 | 91.2 |
| 3b (counter- | | x | | 360 | 91.6 | 79.5 | 86.8 |
| example) | | | | | | | |
| 4a | x | | 380 | 390 | 89.2 | 82.1 | 92.1 |
| 4b (counter- | | x | | 390 | 92.8 | 77.6 | 83.6 |
| example) | | | | | | | |

It has thus been shown clearly that the two-stage process regime (experiments 1a, 2a, 3a and 4a) can achieve higher butadiene yields compared to the one-stage process regime (experiments 1b, 2b, 3b and 4b) with otherwise identical process parameters.

### LIST OF REFERENCE NUMERALS

1 isomerization catalyst

2 dehydrogenation catalyst

3 inert bed

4 physical mixture of isomerization catalyst and dehydrogenation catalyst

5 universal catalyst

6 butene mixture provided

7 isomerization arrangement

8 at least partly isomerized butene mixture

9 dehydrogenation arrangement

10 product mixture

11 butadiene

12 butadiene removal

13 residue

14 discharge stream

15 oxygen/steam

16a first universal zone

16b second universal zone

17 reaction zone

18 regeneration zone

19 oxygenous gas

20 offgas

21 fluidized bed reactor

22 conveying device

1. Process for preparing 1,3-butadiene by heterogeneously catalysed oxidative dehydrogenation of n-butene, in which a butene mixture comprising at least 2-butene is provided,
characterized in that
a) the butene mixture provided is subjected to a heterogeneously catalysed isomerization to obtain an at least partly isomerized butene mixture,
b) and in that the at least partly isomerized butene mixture is then subjected to oxidative dehydrogenation.

2. Process according to claim 1,
characterized in that
the isomerization is effected in such a way that 2-butene present in the butene mixture provided is isomerized to 1-butene, such that the 1-butene content in the at least partly isomerized butene mixture has increased compared to the butene mixture provided.

3. Process according to claim 1,
characterized in that
the isomerization is effected in such a way that 1-butene present in the butene mixture provided is isomerized to 2-butene, such that the 1-butene content in the at least partly isomerized butene mixture has decreased compared to the butene mixture provided.

4. Process according to claim 1,
characterized in that
the at least partly isomerized butene mixture is subjected to the oxidative dehydrogenation without prior removal of components.

5. Process according to claim 1,
characterized in that
the isomerization is effected in the presence of an isomerization catalyst, and in that the oxidative dehydrogenation is effected in the presence of a dehydrogenation catalyst, and the isomerization catalyst and dehydrogenation catalyst are not identical.

6. Process according to claim 5,
characterized in that
the isomerization catalyst comprises at least two different components, the two components having been mixed with one another or the first component having been applied to the second component.

7. Process according to claim 6,
characterized in that
the first component is an alkaline earth metal oxide, especially selected from the group comprising magnesium oxide, calcium oxide, strontium oxide, barium oxide, and where the proportion by weight of the alkaline earth metal oxide in the overall isomerization catalyst is between 0.5% and 20%.

8. Process according to claim 6,
characterized in that
the second component is aluminium oxide or silicon dioxide or a mixture of aluminium oxide and silicon dioxide or an aluminosilicate.

9. Process according to claims 7,
characterized in that
strontium oxide as first component has been applied to aluminium oxide as second component.

10. Process according to claims 7,
characterized in that
magnesium oxide as first component has been mixed with an aluminosilicate as second component.

11. Process according to claim 5,
characterized in that
the dehydrogenation catalyst used is a bismuth molybdate of the general formula (I):
(Moₐ Bi_{b} Fe_{c} (Co+Ni)_{d} Dₑ E_{f} F_{g} Gₕ Hᵢ) Oₓ (I)
in which
D: at least one of the elements from W, P,
E: at least one of the elements from Li, K, Na, Rb, Cs, Mg, Ca, Ba, Sr,
F: at least one of the elements from Cr, Ce, Mn, V,
G: at least one of the elements from Nb, Se, Te, Sm, Gd, La, Y, Pd, Pt, Ru, Ag, Au,
H: at least one of the elements from Si, Al, Ti, Zr
and
the coefficients a to i represent rational numbers selected from the following ranges, including the specified limits:
a=10 to 12
b=0 to 5
c=0.5 to 5
d=2 to 15
e=0 to 5
f=0.001 to 2
g=0 to 5
h=0 to 1.5
i=0 to 800
and
x is a number which is determined by the valency and frequency of the elements other than oxygen.

12. Process according to claim 5,
characterized in that
the isomerization is effected in an isomerization arrangement of the following specification:
a) the isomerization arrangement comprises a reaction zone and a regeneration zone;
b) the isomerization is effected within the reaction zone of the isomerization arrangement in the presence of isomerization catalyst disposed in the reaction zone of the isomerization arrangement;
c) there is simultaneous regeneration of isomerization catalyst disposed in the regeneration zone of the isomerization arrangement, especially by burning off deposits on the isomerization catalyst with an oxygenous gas;
d) there is continuous exchange of isomerization catalyst between the reaction zone and the regeneration zone of the isomerization arrangement.

13. Process according to claim 5,
characterized in that
the isomerization is effected in an isomerization arrangement of the following specification:
a) the isomerization arrangement comprises two universal zones, each of which is utilizable either as reaction zone or as regeneration zone;
b) one of the two universal zones is utilized as reaction zone for isomerization, while the other universal zone is being utilized as regeneration zone for regeneration of the isomerization catalyst;
c) the isomerization is effected within the universal zone utilized as reaction zone in the presence of isomerization catalyst disposed in the reaction zone;
d) there is simultaneous regeneration of isomerization catalyst disposed in the universal zone utilized as regeneration zone, especially by burning off deposits on the isomerization catalyst with an oxygenous gas.

14. Process according to claim 13,
characterized in that
the respective functions of the universal zones are switched cyclically.

15. Process according to claim 13,
characterized in that
both universal zones are utilized as reaction zones in parallel until a level of deactivation is attained, and in that then one of the two universal zones is utilized as regeneration zone, while the other universal zone continues to be utilized as reaction zone.

16. Process according to claim 1,
in which a product mixture containing 1,3-butadiene is drawn off from the oxidative dehydrogenation and subjected to a butadiene removal, in the course of which 1,3-butadiene is separated from other constituents of the product mixture,
characterized in that
a portion of the product mixture is recycled and blended with the butene mixture provided and/or with the at least partially isomerized butene mixture.

17. Process according to claim 1,
characterized in that
the butene mixture is provided in gaseous form and the isomerization and/or the oxidative dehydrogenation is conducted under the following reaction conditions:
temperature: 250° C. to 500° C., especially 300° C. to 420° C.
pressure: 0.08 to 1.1 MPa, especially 0.1 to 0.8 MPa
weight hourly space velocity (g(butenes)/g(active catalyst composition)/h): 0.1 h⁻¹ to 5.0 h⁻¹, especially 0.15 h⁻¹ to 3.0 h⁻¹

18. Process according to claim 1,
wherein the oxidative dehydrogenation is performed in the presence of steam and oxygen,
characterized in that
steam and/or oxygen is added to the at least partly isomerized butene mixture.

19. Process according to claim 1,
characterized in that
the oxidative dehydrogenation is performed in the presence of an inert gas such as, more particularly, nitrogen and/or steam.

20. Process according to claim 1,
characterized in that
the butene mixture provided has a 1-butene content below the thermodynamic equilibrium concentration of 1-butene that arises from the temperatures that exist in the oxidative dehydrogenation and/or in the isomerization, especially in that the butene mixture provided obeys the following specification:
a) the proportion by weight of hydrocarbons having four carbon atoms, based on the overall butene mixture provided, is at least 90%;
b) the total proportion by weight of n-butane and isobutane, based on the overall butene mixture provided, is 0% to 90%;
c) the total proportion by weight of isobutene, 1-butene, cis-2-butene and trans-2-butene, based on the overall butene mixture provided, is 5% to 100%;
d) the total proportion by weight of cis-2-butene and trans-2-butene, based on the butene content of the butene mixture provided, is 5% to 100%.

21. Process according to claim 20,
characterized in that
the ratio of the 1-butene present in the butene mixture provided to the 2-butene present in the butene mixture provided is subject to variation over time.

22. Process according to claim 21,
characterized in that
the absolute 1-butene and 2-butene contents in the butene mixture provided are subject to variation over time.
